(19) United States
(12) Patent Application Publication
(10) Pub. No.: US 2014/0364774 A1
(43) Pub. Date: Dec. 11, 2014

(54) ULTRASOUND NEUROMODULATION FOR CLINICAL EFFECTS

(71) Applicant: David J. Mishelevich, Playa del Rey, CA (US)

(72) Inventor: David J. Mishelevich, Playa del Rey, CA (US)

(21) Appl. No.: 13/914,929

(22) Filed: Jun. 11, 2013

(51) Int. Cl. A61N 7/00

(52) U.S. Cl. CPC A61N 7/00
USPC 601/2

(57) ABSTRACT

Disclosed are methods and systems and methods for non-invasive neuromodulation using ultrasound to treat Traumatic Brain Injury including Concussion, Compulsive Sexual Behavior (sometimes called Compulsive Sexual Disorder), meningitis, and also provide for the elicitation of emotional catharsis. The neuromodulation can produce acute or long-term effects. The latter occur through Long-Term Depression (LTD) and Long-Term Potentiation (LTP) via training Included is control of direction of the energy emission, intensity, frequency, pulse duration, pulse pattern, and phase/intensity relationships to targeting and accomplishing up regulation and/or down regulation. Use of ultrasound neuromodulation in sessions can enhance the effects.

### CROSS REFERENCE TO RELATED APPLICATIONS

This patent application claims priority to U.S. Provisional Patent Applications No. 61/657,891, titled “ULTRASOUND NEUROMODULATION TREATMENT OF COMPULSIVE SEXUAL BEHAVIOR,” filed on Jun. 11, 2012 and No. 61/666,825, titled “ULTRASOUND NEUROMODULATION DELIVERED IN SESSIONS”, filed on Jun. 30, 2012.

This patent application may be related to one or more of the following patents and pending patent applications (US and PCT applications), each of which is herein incorporated by reference in its entirety: U.S. patent application Ser. No. 12/940,052, titled “NEUROMODULATION OF DEEP-BRAIN TARGETS USING FOCUSED ULTRASOUND,” filed on Nov. 5, 2010; U.S. patent application Ser. No. 12/958,411, titled “MULTI-MODALITY NEUROMODULATION OF BRAIN TARGETS,” filed on Dec. 2, 2010; U.S. patent application Ser. No. 13/007,626, titled “PATIENT FEEDBACK FOR CONTROL OF ULTRASOUND DEEP-BRAIN NEUROMODULATION,” filed on Jan. 15, 2011; U.S. patent application Ser. No. 13/200,903, titled “SHAPED AND STEERED ULTRASOUND FOR DEEP-BRAIN NEUROMODULATION,” filed on Jan. 15, 2011; U.S. patent application Ser. No. 13/098,473, titled “ULTRASOUND MACRO-PULSE AND MICRO-PULSE SHAPES FOR NEUROMODULATION,” filed on May 1, 2011; U.S. patent application Ser. No. 13/252,054, titled “ULTRASOUND-INTERSECTING BEAMS FOR DEEP-BRAIN NEUROMODULATION,” filed on Oct. 3, 2011; and U.S. patent application Ser. No. 13/360,600, titled “PATTERNED CONTROL OF ULTRASOUND FOR NEUROMODULATION,” filed on Jan. 27, 2012.

### INCORPORATION BY REFERENCE

All publications, including patents and patent applications, mentioned in this specification are herein incorporated by reference in their entirety to the same extent as if each individual publication was specifically and individually cited to be incorporated by reference.

### FIELD OF THE INVENTION

Described herein are systems and methods for Ultrasound Neuromodulation including one or more ultrasound sources for neuromodulation of target deep brain regions to up-regulate or down-regulate neural activity to obtain a clinical effect.

### BACKGROUND OF THE INVENTION

It has been demonstrated that focused ultrasound directed at neural structures can stimulate those structures. If neural activity is increased or excited, the neural structure is up regulated; if neural activated is decreased or inhibited, the neural structure is down regulated. Neural structures are usually assembled in circuits. For example, nuclei and tracts connecting them make up a circuit. The potential application of ultrasonic therapy of deep-brain structures has been suggested previously (Gavrilov L R, Tsirulnikov E M, and I A Davies, “Application of focused ultrasound for the stimulation of neural structures,” Ultrasound Med Biol. 1996; 22(2):179-92. and S. J. Norton, “Can ultrasound be used to stimulate nerve tissue?,” BioMedical Engineering OnLine 2003, 2:6). Norton notes that while Transcranial Magnetic Stimulation (TMS) can be applied within the head with greater intensity, the gradients developed with ultrasound are comparable to those with TMS. It was also noted that monophasic ultrasound pulses are more effective than biphasic ones. Instead of using ultrasonic stimulation alone, Norton applied a strong DC magnetic field as well and describes the mechanism as that given that the tissue to be stimulated is conductive that particle motion induced by an ultrasonic wave will induce an electric current density generated by Lorentz forces.

The effect of ultrasound is at least two fold. First, increasing temperature will increase neural activity. An increase up to 42 degrees C. (say in the range of 39 to 42 degrees C.) locally for short time periods will increase neural activity in a way that one can do so repeatedly and be safe. One needs to make sure that the temperature does not rise about 50 degrees C. or tissue will be destroyed (e.g., 56 degrees C. for one second). This is the objective of another use of therapeutic application of ultrasound, ablation, to permanently destroy tissue (e.g., for the treatment of cancer). An example is the ExAblate device from InSightec in Haifa, Israel. The second mechanism is mechanical perturbation. An explanation for this has been provided by Tyler et al. from Arizona State University (Tyler, W. J., Y. Tufail, M. Finsterwald, M. L. Tauchmann, E. J. Olsen, C. Majestic, “Remote excitation of neuronal circuits using low-intensity, low-frequency ultrasound,” PLoS One 3(10): e3511, doi:10.137/1/journal.pone.0003511, 2008)) where voltage gating of sodium channels in neural membranes was demonstrated. Pulsed ultrasound was found to cause mechanical opening of the sodium channels that resulted in the generation of action potentials. Their stimulation is described as Low Intensity Low Frequency Ultrasound (LILFU). They used bursts of ultrasound at frequencies between 0.44 and 0.67 MHz, lower than the frequencies used in imaging. Their device delivered 23 milliwatts per square centimeter of brain—a fraction of the roughly 180 mW/cm² upper limit established by the U.S. Food and Drug Administration (FDA) for womb-scanning sonograms; thus such devices should be safe to use on patients. Ultrasound impact to open calcium channels has also been suggested. The above approach is incorporated in a patent application submitted by Tyler (Tyler, William, James P., PCT/US2009/050560, WO 2010/009141, published Jan. 21, 2011).

Alternative mechanisms for the effects of ultrasound may be discovered as well. In fact, multiple mechanisms may come into play, but, in any case, this would not effect this invention.

Approaches to date of delivering focused ultrasound vary. Bystritsky (U.S. Pat. No. 7,283,861, Oct. 16, 2007) provides for focused ultrasound pulses (FUP) produced by multiple ultrasound transducers (said preferably to number in the range of 300 to 1000) arranged in a cap place over the skull to affect a multi-beam output. These transducers are coordinated by a computer and used in conjunction with an imaging system, preferable an fMRI (functional Magnetic Resonance Imaging), but possibly a PET (Positron Emission Tomography) or V-EEG (Video-Electroencephalography) device. The user interacts with the computer to direct the FUP to the desired point in the brain, sees where the stimulation actually occurred by viewing the imaging result, and thus adjusts the position of the FUP according. The position of focus is obtained by adjusting the phases and amplitudes of the ultrasound transducers (Clement and Hynynen, “A non-invasive method for focusing ultrasound through the human skull,” Phys. Med. Biol. 47 (2002) 1219-1236). The imaging also illustrates the functional connectivity of the target and surrounding neural structures. The focus is described as two or more centimeters deep and 0.5 to 1000 mm in diameter or preferably in the range of 2-12 cm deep and 0.5-2 mm in diameter. Either a single FUP or multiple FUPs are described as being able to be applied to either one or multiple live neuronal circuits. It is noted that differences in FUP phase, frequency, and amplitude produce different neural effects. Low frequencies (defined as below 500 Hz.) are inhibitory. High frequencies (defined as being in the range of 500 Hz to 5 MHz) are excitatory and activate neural circuits. This works whether the target is gray or white matter. Repeated sessions result in long-term effects. The cap and transducers to be employed are preferably made of non-ferrous material to reduce image distortion in fMRI imaging. It was noted that if after treatment the reactivity as judged with fMRI of the patient with a given condition becomes more like that of a normal patient, this may be indicative of treatment effectiveness. The FUP is to be applied 1 ms to 1 s before or after the imaging. In addition a CT (Computed Tomography) scan can be run to gauge the bone density and structure of the skull.

Deisseroth and Schneider (U.S. patent application Ser. No. 12/263,026 published as US 2009/0112133 A1, Apr. 30, 2009) describe an alternative approach in which modifications of neural transmission patterns between neural structures and/or regions are described using ultrasound (including use of a curved transducer and a lens) or RF. The impact of Long-Term Potentiation (LTP) and Long-Term Depression (LTD) for durable effects is emphasized. It is noted that ultrasound produces stimulation by both thermal and mechanical impacts. The use of ionizing radiation also appears in the claims.

Adequate penetration of ultrasound through the skull has been demonstrated (Hynynen, K. and F A Jolesz, “Demonstration of potential noninvasive ultrasound brain therapy through an intact skull,” Ultrasound Med Biol, 1998 February; 24(2):275-83 and Clement G T, Hynynen K (2002) A non-invasive method for focusing ultrasound through the human skull. Phys Med Biol 47: 1219-1236.). Ultrasound can be focused to 0.5 to 2 mm as TMS to 1 cm at best.

Because of the utility of ultrasound in the neuromodulation of deep-brain structures, it would be both logical and desirable to apply it to the treatment of Traumatic Brain Injury, including concussion, Compulsive Sexual Behavior (sometimes called Compulsive Sexual Disorder), and also the elicitation of emotional catharsis.

### SUMMARY OF THE INVENTION

It is the purpose of this invention to provide methods and systems for non-invasive neuromodulation using ultrasound to treat multiple conditions, including Traumatic Brain Injury (TBI) including concussion, Compulsive Sexual Behavior (sometimes called Compulsive Sexual Disorder), and also provide for the elicitation of emotional catharsis. Such neuromodulation can produce acute effects or Long-Term Potentiation (LTP) or Long-Term Depression (LTD). Effectiveness of the treatment can be enhanced by the application of the ultrasound neuromodulation in sessions. An example of sessions is neuromodulation for 30 minutes per day for five days a week over a two-week period. Most applications will not be amenable to continuous neuromodulation. Application of patterned ultrasound neuromodulation can enhance the effects.

Included is control of direction of the energy emission, intensity, frequency (carrier frequency and/or neuromodulation frequency), pulse duration, pulse pattern, and phase/intensity relationships to targeting and accomplishing up-regulation and/or down-regulation. Use of ancillary monitoring or imaging to provide feedback is optional. In embodiments where concurrent imaging is performed, the device of the invention is constructed of non-ferrous material.

To accomplish the treatment, in some cases the neural targets will be up regulated and in some cases down regulated, depending on the given neural target. Targets have been identified by such methods as PET imaging, fMRI imaging, and clinical response to Deep-Brain Stimulation (DBS) or Transcranial Magnetic Stimulation (TMS).

The most common areas for focal damage for Traumatic Brain Injury for non-penetrating lesions are the Orbito-Frontal Cortex (OFC) (Mattson, A. J.; Levin, H. S. (1990). “Frontal lobe dysfunction following closed head injury; a review of the literature,” Journal of Nervous & Mental Disorders 178 (5): 282-291. doi:10.1097/00005053-199005000-00002) and the Occipital Lobe (Bayly, P. V.; Cohen, T. S., Leister, E. P., Ajo, D., Leuthardt, E. C., & G M Genin, “Deformation of the human brain induced by mild acceleration,” Journal of Neurotrauma 22 (8): 845-856, 2005. doi:10.1089/neu.2005.22.845. PMC 2377024. PMID 16083352). For milder injuries (e.g., concussions), areas involved are varied and include such regions as the Thalamus and Hypothalamus (together the diencephalon) the Reticular Activating, midbrain, Fornix, Temporal Lobe, Frontal Lobe, as well the corpus collosum, midbrain, and brainstem (e.g., Bigler E D, “Neuropsychology and clinical neuroscience of persistent post-concussive syndrome,” Journal of the International Neuropsychological Society 14 (1): 1-22, 2008 and Ropper A H, and K C Gorson, “Clinical practice. Concussion,” New England Journal of Medicine 356 (2): 166-72, 2007. doi:10.1056/NEJMcp064645. PMID 17215534).

As to targets for Compulsive Sexual Behavior (e.g., Hilton D L, Watts C. Pornography addiction: A neuroscience perspective. Surg Neurol Int 2011; 2:19), the Medial Pre-Frontal Cortex, Nucleus Accumbens, Hypothalamus, and Ventral Tegmental Area are down regulated. The Amygdala can be involved as well (Ochsner, K N, & L F Barrett, “A multiprocess perspective on the neuroscience of emotion,” in T. J. Mayne & G. A. Bonanno (Eds.), Emotions: Current issues and future directions (pp. 38-81), 2001 New York: Guilford). Frontal cortical dysfunction is related to improper serotonin processing (decreased serotonin or blunted response to serotonin). Nucleus Accumbens dysfunction is due to dopamine release. Targets depend on specific patients and relationships among the targets. Prevalence for the condition is a little less than 5%. Treatment for conditions (e.g., Parkinson's Disease) may cause Compulsive Sexual Behavior. In some cases neuromodulation will be bilateral and in others unilateral. The specific targets and/or whether the given target is up regulated or down regulated, can depend on the individual patient and relationships of up regulation and down regulation among targets, and the patterns of stimulation applied to the targets.

Elicitation of catharsis depends on triggering of emotion that is most effectively accomplished by neuromodulating the limbic system. Typidal targets are the Amygdala and the Hippocampus (S E Morrison and C D Salzman, “Re-valuing the Amygdala,” Current Opin. Neurobiol. 2010 April; 20(2): 221-230. doi:10.1016/j.conb.2010.02.007. The Pre-Frontal Cortex (PFC) can be another target for neuromodulation.

Effectiveness of the treatment can be enhanced by the application of the ultrasound neuromodulation in sessions. An example of sessions is neuromodulation for 30 minutes per day for five days a week over a two-week period. Most applications will not be amenable to continuous neuromodulation. Application of patterned ultrasound neuromodulation can enhance the effects.

Targets depend on specific patients and relationships among the targets. In some cases neuromodulation will be bilateral and in others unilateral. The specific targets and/or whether the given target is up regulated or down regulated, can depend on the individual patient and relationships of up regulation and down regulation among targets, and the patterns of stimulation applied to the targets.

The targeting can be done with one or more of known external landmarks, an atlas-based approach or imaging (e.g., fMRI or Positron Emission Tomography). The imaging can be done as a one-time set-up or at each session although not using imaging or using it sparingly is a benefit, both functionally and the cost of administering the therapy, over Bystritsky (U.S. Pat. No. 7,283,861) which teaches consistent concurrent imaging.

While ultrasound can be focused down to a diameter on the order of one to a few millimeters (depending on the frequency), whether such a tight focus is required depends on the conformation of the neural target.

### BRIEF DESCRIPTION OF THE DRAWINGS

FIG. 1 shows a diagram of exemplar session types for both initial treatment and maintenance sessions.

FIG. 2 shows a block diagram of the control circuit.

FIG. 3 shows ultrasonic-transducer targeting of the Orbito-Frontal Cortex (OFC) and the Occipital Lobe for the treatment of Traumatic Brain Injury.

FIG. 4 shows ultrasound-transducer targeting of the Orbito-Frontal Cortex (OFC), Temporal Lobe, Thalamus, Hypothalamus, and Fornix for the treatment of concussion.

FIG. 5 shows ultrasonic-transducer targeting of the Medial Pre-Frontal Cortex, Nucleus Accumbens, Hypothalamus, and Ventral Tegmental Area for the treatment of Compulsive Sexual Behavior.

FIG. 6 shows ultrasound-transducer targeting of the Amygdala and Hippocampus for the elicitation of emotional catharsis.

### DETAILED DESCRIPTION OF THE INVENTION

It is the purpose of this invention to provide methods and systems and methods for neuromodulation of deep-brain targets using ultrasound to treat Traumatic Brain Injury including concussion, Compulsive Sexual Behavior (sometimes called Compulsive Sexual Disorder), and also provide for the elicitation of emotional catharsis. In addition, effectiveness of the treatment can be enhanced by the application of the ultrasound neuromodulation in sessions and also the use of stimulation patterns. Most applications will not be amenable to continuous neuromodulation.

With respect to delivery of ultrasound neuromodulation in sessions, examples of session types include periodic sessions over extended time typically means a single session of length on the order of 15 to 60 minutes repeated daily or five days per week over one to six weeks. Other lengths of session or number of weeks of neuromodulation are applicable, such as session lengths up to 2.5 hours and number of weeks ranging from one to eight. Period sessions over compressed time typically means a single session of length on the order of 30 to 60 minutes repeated during awake hours with inter-session times of 15 minutes to 60 minutes over one to three days. Other inter-session times such as 15 minutes to three hours and days of compressed therapy such as one to five days are applicable. Maintenance consists of periodic sessions at fixed intervals or on as-needed maintenance tune-ups. Maintenance categories are maintenance post-completion of original treatment at fixed intervals and maintenance post-completion of original treatment with as-needed maintenance tune-ups. An example of the former are with one or more 50-minutes sessions during week 2 of months four and eight, and of the latter is one or more 50-minute sessions during week 7 because a tune up is needed at that time as indicated by return of symptoms. Use of sessions is important for the retraining of neural pathways for change of function, maintenance of function, or restoration of function. Retraining over time, with its ongoing reinforcement, can allow more effectively achievement of desired impacts. Another consideration is the desirability for practical reasons to limit tying up the time of the patient depending on the individual situation. Such neuromodulation systems can produce applicable acute or long-term effects. The latter occur through Long-Term Depression (LTD) or Long-Term Potentiation (LTP) via training Included is control of direction of the energy emission, intensity, frequency (carrier frequency and/or neuromodulation frequency), pulse duration, pulse pattern, and phase/intensity relationships to targeting and accomplishing up-regulation and/or down-regulation.

The stimulation frequency for inhibition is lower than 500 Hz (depending on condition and patient). The stimulation frequency for excitation is in the range of 500 Hz to 5 MHz. In this invention, the ultrasound acoustic frequency is in range of 0.3 MHz to 0.8 MHz with power generally applied less than 60 mW/cm² but also at higher target- or patient-specific levels at which no tissue damage is caused. The acoustic frequency is modulated at the lower rate to impact the neuronal structures as desired (e.g., say typically 300 Hz for inhibition (down-regulation) or 1 kHz for excitation (up-regulation). The modulation frequency (superimposed on the carrier frequency of say 0.5 MHz or similar) may be divided into pulses 0.1 to 20 msec. repeated at frequencies of 2 Hz or lower for down regulation and higher than 2 Hz for up regulation although this will be both patient and condition specific. Ultrasound therapy can be combined with therapy using other devices (e.g., Transcranial Magnetic Stimulation (TMS)).

The lower bound of the size of the spot at the point of focus will depend on the ultrasonic frequency, the higher the frequency, the smaller the spot. Ultrasound-based neuromodulation operates preferentially at low frequencies relative to say imaging applications so there is less resolution. Keramos-Etalon can supply a 1-inch diameter ultrasound transducer and a focal length of 2 inches that with 0.4 Mhz excitation will deliver a focused spot with a diameter (6 dB) of 0.29 inches. Typically, the spot size will be in the range of 0.1 inch to 0.6 inch depending on the specific indication and patient. A larger spot can be obtained with a 1-inch diameter ultrasound transducer with a focal length of 3.5″ which at 0.4 MHz excitation will deliver a focused spot with a diameter (6 dB) of 0.51.″ Even though the target is relatively superficial, the transducer can be moved back in the holder to allow a longer focal length. Other embodiments are applicable as well, including different transducer diameters, different frequencies, and different focal lengths. Other ultrasound transducer manufacturers are Blatek and Imasonic. In an alternative embodiment, focus can be deemphasized or eliminated with a smaller ultrasound transducer diameter with a shorter longitudinal dimension, if desired, as well. Ultrasound conduction medium will be required to fill the space.

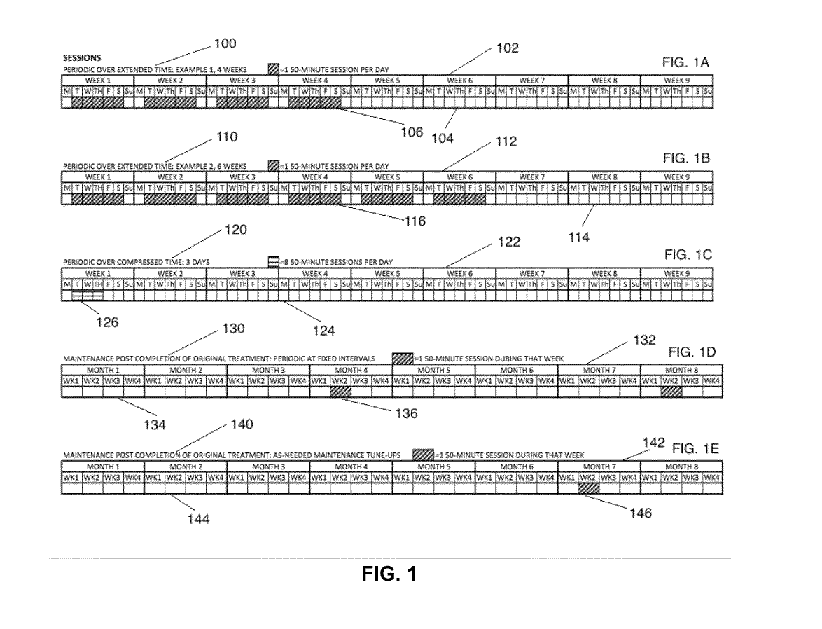

FIG. 1 shows a diagram of exemplar session types for both initial treatment and maintenance sessions. FIG. 1A illustrates example 100, Periodic Over Extended Time with 4 weeks of treatment where time divisions are weeks 102 divided into days 104 with 50-minute sessions on indicated days 106. For all of these examples, the session length could be longer or shorter than 50 minutes. FIG. 1B illustrates example 110, Periodic Over Extended Time with 6 weeks of treatment where time divisions are weeks 112 divided into days 114 with 50-minute sessions on indicated days 116. FIG. 1C illustrates example 120, Periodic Over Compressed Time with 3 days of treatment where time divisions are weeks 122 divided into days 124 with 50-minute sessions on indicated days 166. FIG. 1D illustrates example 130, Maintenance Post Completion of Original Treatment at Fixed Intervals where time divisions are months 132 divided into weeks 134 with 50-minute sessions during indicated weeks 136. FIG. 1E illustrates example 140, Maintenance Post Completion of Original Treatment with As-Needed Maintenance Tune-Ups where time divisions are months 142 divided into weeks 144 with 50-minute sessions during indicated week 146.

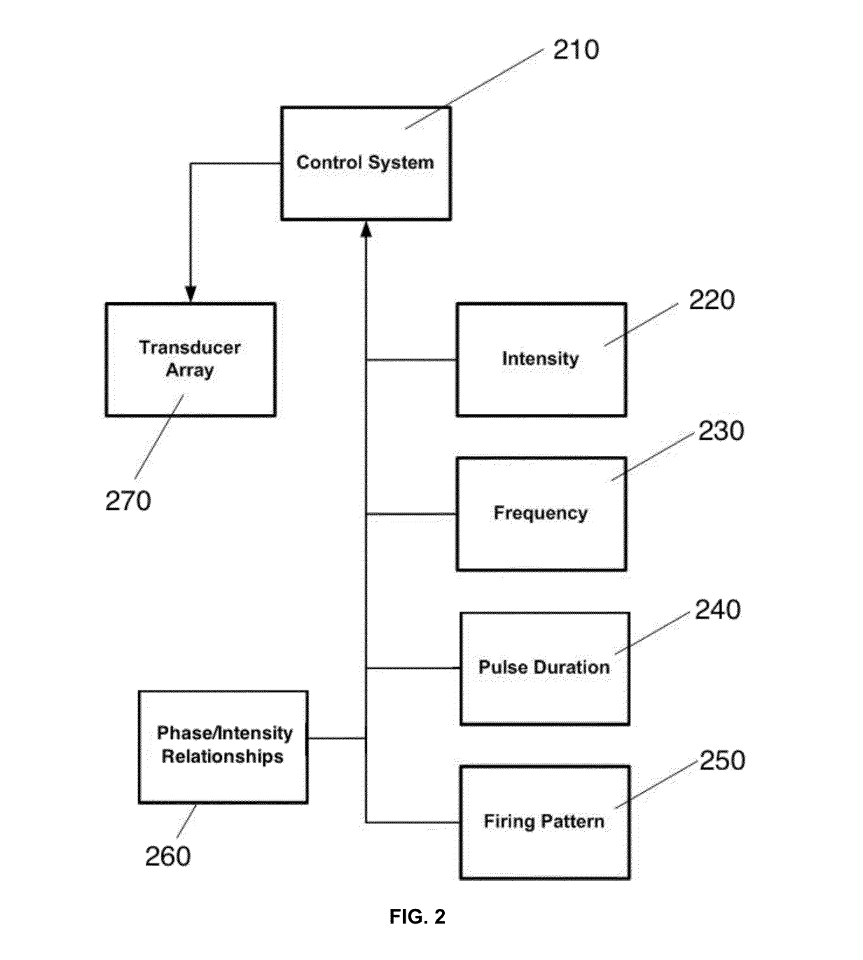

FIG. 2 shows an embodiment of a control circuit. The positioning and emission characteristics of transducer array 270 are controlled by control system 210 with control input with neuromodulation characteristics determined by settings of intensity 220, frequency 230, pulse duration 240, firing pattern 250, and phase/intensity relationships 260 for beam steering and focusing on neural targets.

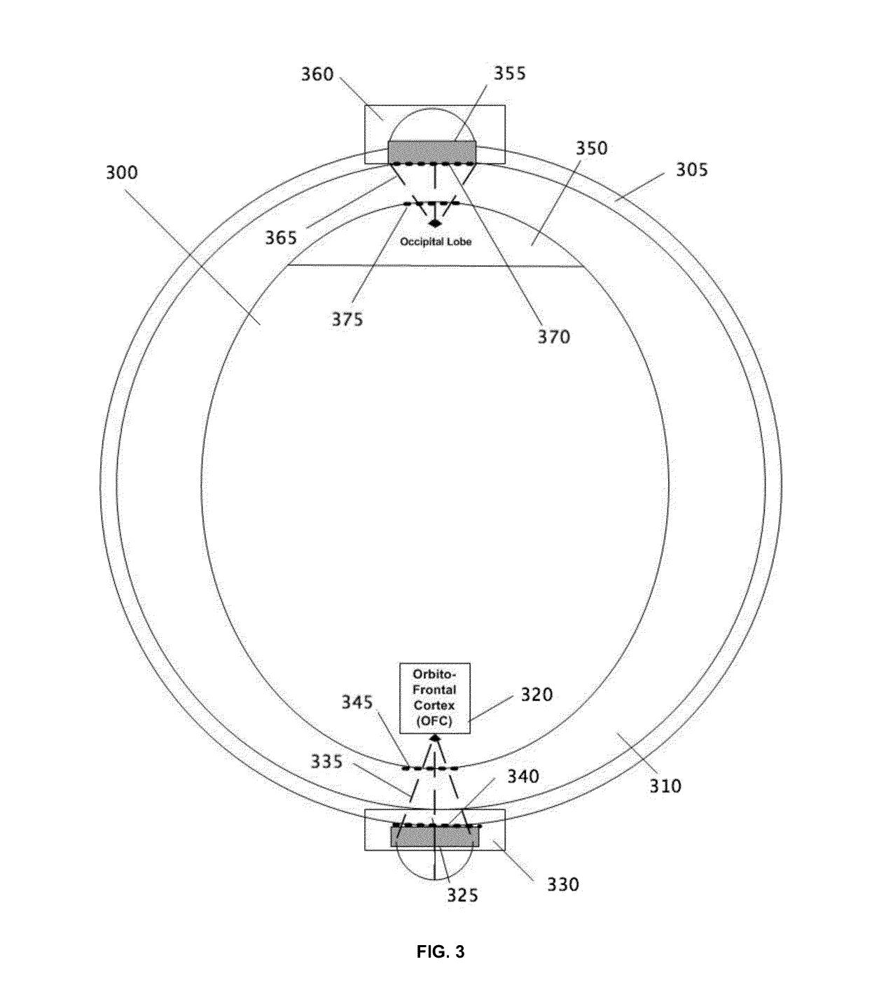

FIG. 3 shows a set of ultrasound transducers targeting to treat Traumatic Brain Injury. Head 300 contains two targets, Orbital-Frontal Cortex (OFC) 320 and Occipital Lobe 350, both of which are to be up regulated. The Orbito-Frontal Cortex in this context represents itself and other regions of the Frontal Lobe. While two targets are involved here, others might work as well, or an addition or substitution of other targets. The targets shown are hit by ultrasound from transducers 325 (in carrier 330) and 355 (in carrier 360) fixed to track 305. Ultrasound transducer 325 with its beam 335 is shown targeting Orbito-Frontal Cortex (OFC) 320 and transducer 355 with its beam 365 is shown targeting Occipital Lobe 350. For ultrasound to be effectively transmitted to and through the skull and to brain targets, coupling must be put into place. Ultrasound transmission (for example Dermasol from California Medical Innovations) medium 310 is interposed with one mechanical interface to the frame 305 and ultrasound transducers 325 and 355 (completed by a layers of ultrasound transmission gel layers 340 and 370 respectively) and the other mechanical interface to the head 300 (completed by a layers of ultrasound transmission gel 345 and 375 respectively). In another embodiment the ultrasound transmission gel is placed around the entire frame and entire head. In another embodiment, multiple ultrasound transducers whose beams intersect at that target replace an individual ultrasound transducer for that target. In still another embodiment, mechanical perturbations are applied radially or axially to move the ultrasound transducers.

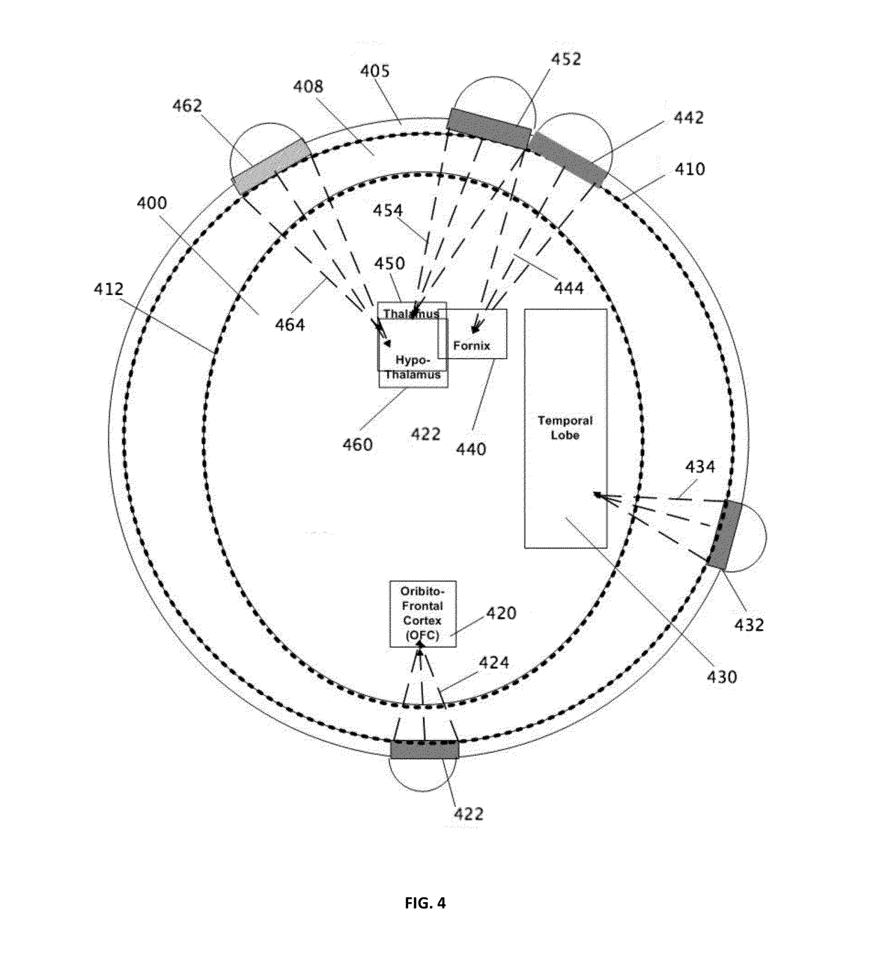

FIG. 4 shows a set of ultrasound transducers targeting to treat concussion. Head 400 contains five targets, Orbital-Frontal Cortex (OFC) 420, Temporal Lobe 430, Fornix 440, Thalamus 450, and Hypothalamus 460, all of which are to be up regulated. The Orbito-Frontal Cortex in this context represents itself and other regions of the Frontal Lobe. While five targets are involved here, others might work as well, or an addition or substitution of other targets (e.g., midbrain, Reticular Activating System, brainstem, and corpus callosum) identified currently or in the future. The targets shown are hit by ultrasound from transducers 422, 432, 442, 452, and 462 fixed to track 405. Ultrasound transducer 422 with its beam 424 is shown targeting Orbito-Frontal Cortex (OFC) 420, transducer 432 with its beam 434 is shown targeting Temporal Lobe 430, transducer 442 with its beam 444 is shown targeting Fornix 440, transducer 452 with its beam 454 is shown targeting Thalamus 450, and transducer 462 with its beam 462 is shown targeting Hypothalamus 460. For ultrasound to be effectively transmitted to and through the skull and to brain targets, coupling must be put into place. Ultrasound transmission (for example Dermasol from California Medical Innovations) medium 408 is interposed with one mechanical interface to the frame 205 and ultrasound transducers 422, 432, 442, 452 and 462 (completed by a layer of ultrasound transmission gel layer 410) and the other mechanical interface to the head 400 (completed by a layer of ultrasound transmission gel 412). In another embodiment the ultrasound transmission gel is only placed at the particular places where the ultrasonic beams from the transducers are located rather than around the entire frame and entire head. In another embodiment, multiple ultrasound transducers whose beams intersect at that target replace an individual ultrasound transducer for that target. In still another embodiment, mechanical perturbations are applied radially or axially to move the ultrasound transducers.

Transducer array assemblies of this type may be supplied to custom specifications by Imasonic in France (e.g., large 2D High Intensity Focused Ultrasound (HIFU) hemispheric array transducer) (Fleury G., Berriet, R., Le Baron, O., and B. Huguenin, “New piezocomposite transducers for therapeutic ultrasound,” 2^{nd} International Symposium on Therapeutic Ultrasound—Seattle—Jul. 31-Aug. 2, 2002), typically with numbers of ultrasound transducers of 300 or more. Keramos-Etalon in the U.S. is another custom-transducer supplier. The power applied will determine whether the ultrasound is high intensity or low intensity (or medium intensity) and because the ultrasound transducers are custom, any mechanical or electrical changes can be made, if and as required. At least one configuration available from Imasonic (the HIFU linear phased array transducer) has a center hole for the positioning of an imaging probe. Keramos-Etalon also supplies such configurations.

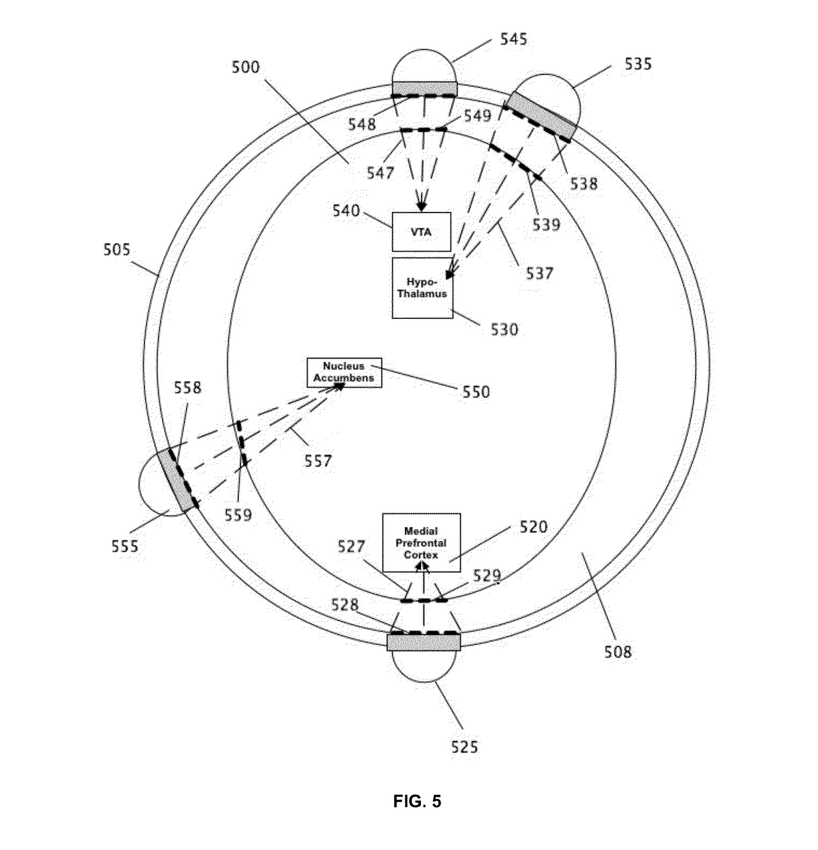

FIG. 5 shows ultrasonic-transducer targeting of the Medial Prefrontal Cortex, Nucleus Accumbens, Hypothalamus, and Ventral Tegmental Area for the treatment of Compulsive Sexual Behavior. A potential additional target is the Amygdala (not shown). Head 100 contains four targets, Medial Prefrontal Cortex 520, Hypothalamus 530, VTA (Ventral Tegmental Area) 540, and Nucleus Accumbens 550, all of which are to be down regulated. While four targets are involved here, others (e.g., Amygdala) might work as well, or an addition or substitution for other targets. The targets shown are hit by ultrasound from transducers 525, 535, 545, and 555 fixed to track 505. Ultrasound transducer 525 with its ultrasound beam 527 is shown targeting Medial Prefrontal Cortex 520, ultrasound transducer 535 with its beam 537 is shown targeting Hypothalamus 530, ultrasound transducer 545 with its beam 547 is shown targeting VTA 540, and transducer 555 with its beam 557 is shown targeting Nucleus Accumbens 550. For ultrasound to be effectively transmitted to and through the skull and to brain targets, coupling must be put into place. Ultrasound transmission (for example Dermasol from California Medical Innovations) medium 508 is interposed with one mechanical interface to the frame 505 and ultrasound transducers 525, 535, 545, and 555 (completed by a layers of ultrasound transmission gel layers 528, 538, 548, 558 respectively) and the other mechanical interface to the head 500 (completed by a layers of ultrasound transmission gel 529, 539, 549, and 559 respectively). In another embodiment the ultrasound transmission gel is placed around the entire frame and entire head. In another embodiment, multiple ultrasound transducers whose beams intersect at that target replace an individual ultrasound transducer for that target. In still another embodiment, mechanical perturbations are applied radially or axially to move the ultrasound transducers.

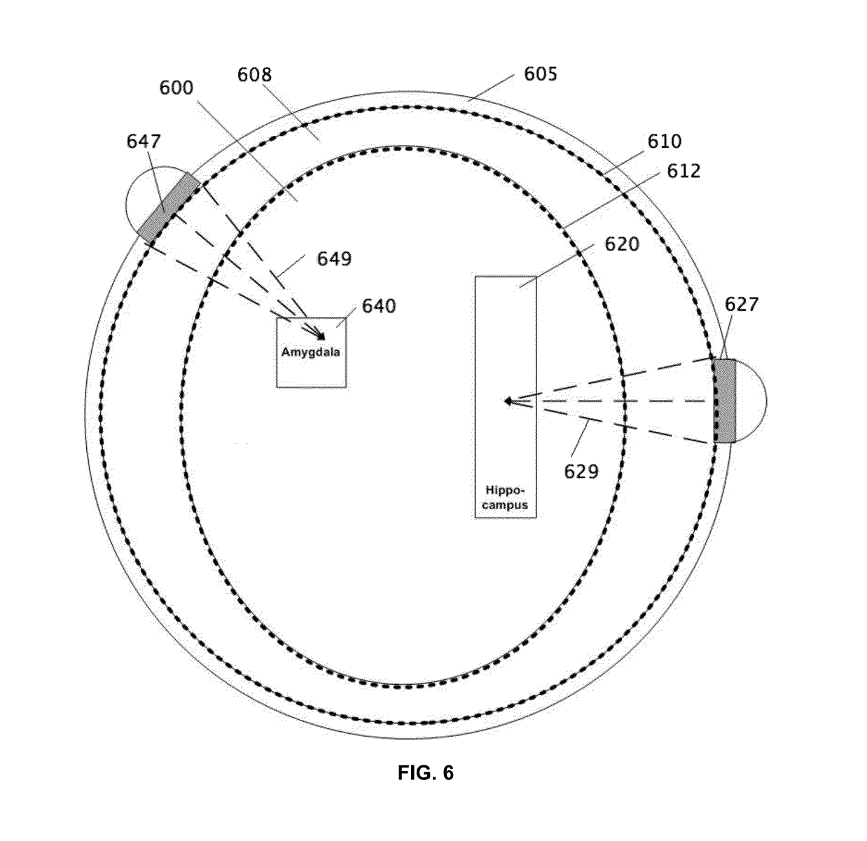

FIG. 6 shows a set of ultrasound transducers targeting to generate an emotional catharsis. Head 600 contains two targets, Hippocampus 620, and Amygdala 640. Both are typically up regulated. Note that while these two targets are covered here, one might work as well, or an addition or substitution of other targets (e.g., thalamus, sub-thalamic nuclei, and basal ganglia) identified currently or in the future. These targets are hit by ultrasound from transducers 627 and 647 fixed to track 605. Ultrasound transducer 627 with its beam 629 is shown targeting hippocampus 620, and transducer 647 with its beam 649 is shown targeting amygdala 640. Bilateral stimulation of one of a plurality of these targets is another embodiment. The Pre-Frontal Cortex (PFC) is potentially another target.

In another embodiment, a feedback mechanism is applied such as functional Magnetic Resonance Imaging (fMRI), Positive Emission Tomography (PET) imaging, video-electroencephalogram (V-EEG), acoustic monitoring, thermal monitoring, and patient feedback.

In still other embodiments, other energy sources are used in combination with or substituted for ultrasound transducers that are selected from the group consisting of Transcranial Magnetic Stimulation (TMS), deep-brain stimulation (DBS), optogenetics application, radiosurgery, Radio-Frequency (RF) therapy, behavioral therapy, and medications.

The invention allows stimulation adjustments in variables such as, but not limited to, direction of the energy emission, intensity, frequency (carrier frequency and/or neuromodulation frequency), pulse duration, pulse pattern, and phase/intensity relationships to targeting and accomplishing up-regulation and/or down-regulation, dynamic sweeps, and position.

The various embodiments described above are provided by way of illustration only and should not be construed to limit the invention. Based on the above discussion and illustrations, those skilled in the art will readily recognize that various modifications and changes may be made to the present invention without strictly following the exemplary embodiments and applications illustrated and described herein. Such modifications and changes do not depart from the true spirit and scope of the present invention.

What I claim is:

1. A method of non-invasively neuromodulating one or a plurality of targets using ultrasound stimulation, the method comprising:
aiming one or a plurality of ultrasound transducers at one or a plurality of targets,
applying pulsed power to said ultrasound transducers via a control circuit thereby modulating the activity of the targets, and
applying the neuromodulation in sessions,
whereby therapeutic results are obtained where the targets are related to treatment of traumatic brain injury including concussion, compulsive sexual behavior, or the elicitation of emotional catharsis.

2. The method of claim 1, wherein the one or plurality of neural targets for the treatment of traumatic brain injury are selected from the group consisting of Orbito-Frontal Cortex and Occipital Lobe.

3. The method of claim 1, wherein the one or plurality of neural targets for the treatment of concussion are selected from the group consisting of Orbital-Frontal Cortex, Temporal Lobe, Fornix, Thalamus, and Hypothalamus.

4. The method of claim 1, wherein the one or plurality of neural targets for the treatment of compulsive sexual behavior is selected from the group consisting of Medial Prefrontal Cortex, Hypothalamus, Ventral Tegmental Area, Nucleus Accumbens, and Amygdala.

5. The method of claim 1 further comprising aiming an ultrasound transducer neuromodulating neural targets in a manner selected from the group of up-regulation, down-regulation.

6. The method of claim 1 wherein the effect is chosen from the group consisting of acute, Long-Term Potentiation, and Long-Term Depression.

7. The method of claim 1 wherein a feedback mechanism is applied, wherein the feedback mechanism is selected from the group consisting of functional Magnetic Resonance Imaging (fMRI), Positive Emission Tomography (PET) imaging, video-electroencephalogram (V-EEG), acoustic monitoring, thermal monitoring, patient.

8. The method of claim 1 wherein ultrasound therapy is combined with or replaced by one or more therapies selected from the group consisting of Transcranial Magnetic Stimulation (TMS), deep-brain stimulation (DBS), application of optogenetics, radiosurgery, Radio-Frequency (RF) therapy, behavioral therapy, and medications.
